(19) United States
(12) Patent Application Publication
(10) Pub. No.: US 2011/0033504 A1
(43) Pub. Date: Feb. 10, 2011

(54) ARTICLES AND METHODS FOR REPAIRING DAMAGED NERVOUS TISSUE

(75) Inventors: Margaret Wheatley, Media, PA (US); Justin Lathia, Baltimore, MD (US); Mihir Shanbhag, Manalapan, NJ (US); Nicola Francis, Hamilton Parish (BM)

Correspondence Address: WOODCOCK WASHBURN LLP, CIRA CENTRE, 12TH FLOOR, 2929 ARCH STREET, PHILADELPHIA, PA, 19104-2891, US

(73) Assignee: DREXEL UNIVERSITY, Philadelphia, PA (US)

(21) Appl. No.: 12/670,173

(22) PCT Filed: Jul. 23, 2008
(86) PCT No.: PCT/US08/70849

§ 371 Date: Apr. 22, 2010

Related U.S. Application Data
(60) Provisional application No. 60/951,311, filed on Jul. 23, 2007.
(60) Provisional application No. 61/023,787, filed on Jan. 25, 2008.

(51) Int. Cl. A61K 35/30; C12N 5/0775; C12N 5/0797; A61K 35/12; A61K 9/00; C12N 5/071; A61P 25/00

(52) U.S. Cl. 424/400; 435/325; 424/93.7

(57) ABSTRACT

The invention concerns substrates comprising a biocompatible gel and at least one of a plurality of cells, said cells being capable of producing at least one therapeutic agent. Other aspects of the invention concern methods of making such substrates and methods of repairing and regenerating damaged nervous tissue with the substrates.

### CROSS-REFERENCE TO RELATED APPLICATIONS

This application claims the benefit of U.S. Provisional Application No. 60/951,311, filed Jul. 23, 2007, and U.S. Provisional Application No. 60/023,787, filed Jan. 25, 2008, the disclosures of which are incorporated herein by reference in their entireties.

### FIELD OF THE INVENTION

The invention concerns articles and methods for repairing damaged nervous tissue utilizing a biocompatible gel containing one or more cells and providing at least one therapeutic agent that originates from said cells or independent from said cells as a separate feature of the described article.

### BACKGROUND OF THE INVENTION

Various publications, including patents, published applications, technical articles and scholarly articles are cited throughout the specification. Each of these cited publications is incorporated by reference herein, in its entirety

Injury to nervous tissue is particularly devastating due to inflammation, scarring, and limited self-regenerative capacity of the tissue. Bioengineered therapies, such as delivering growth factors and/or cells, have become popular due to their potential for repairing damaged tissue. Nomura, et al., J Neurotrauma, 2006. 23(3-4): p. 496-507; Pearse and Bunge, J Neurotrauma, 2006. 23(3-4): p. 438-52; Tuszynski, M. H., et al., Exp Neurol, 2003. 181(1): p. 47-56.

Delivery of neurotrophic factors, such as brain-derived neurotrophic factor (BDNF) and neurotrophic factor-3 (NT3), to damaged nervous tissue has been shown not only to help to prevent neuronal death but also to aid in their regeneration. Tuszynski, supra; Katz and Meiri, J Neurosci, 2006. 26(15): p. 3899-907; Hapner, et al, Dev Biol, 2006 297(1):182-197. A major obstacle to using neurotrophic factors, however, is that they rapidly degrade in vivo, necessitating repeated invasive techniques for their successful administration. Likewise, in vivo degradation would also be an obstacle for delivering peptides, like Humanin, a recently described 24 amino acid linear peptide implicated in preventing cell death in the brain, and potentially of use in Alzheimers treatment . Y. Hashimoto, et al., Proc. Natl. Acad. Sci. U.S.A, 2001.98: p 6336; Y. Hashimoto, et al. Biochem. and Biophys. Res. Comm., 2001.283, p 460-468. To overcome this obstacle, researchers have attempted to transplant genetically modified fibroblasts that continually produce neurotrophic factors. Nakahara, et al. Cell Transplant, 1996. 5(2): p. 191-204; Kim, et al., Neuroreport, 1996. 7(13): p. 2221-5; Jin, et al., Neurorehabil Neural Repair, 2000. 14(4): p. 311-7; Tobias, et al., J Neurotrauma, 2001. 18(3): p. 287-301. Such transplantation, however, may trigger deleterious immune responses that ultimately limit the effectiveness of this approach.

Even if transplanting growth factor- or peptide-producing cells is effective, growth factors and peptides may not be able to penetrate damaged tissue, again necessitating repeated invasive techniques for successful administration. Similarly, damaged tissue may not support the regeneration or growth of cellular structures, such as axons, dendrites, or myelin. Thus there is a need to make the damage tissue permissive for growth factor diffusion and/or cellular repair, regeneration, and growth.

Replacing lost or injured cells using stem cells, such as neural progenitor cells (NPC's), is another approach to repairing the damaged tissue. In addition to facing the same obstacles as other transplanted cells, NPCs must differentiate to particular cell types to serve as suitable replacements for lost or non-functioning cells. Also, NPCs must be present at particular locations to replace lost cells. There is a need to direct NPCs differentiation and direct the migration of differentiated cells. Additionally, NPCs may act as a source of therapeutic agent to make the damaged tissue permissive for growth factor diffusion and/or cellular repair, regeneration, and growth.

Thus, there is a need for articles and methods to repair damaged nervous tissue by delivering growth factors, peptides, and/or cells capable of replacing lost cells, as well as delivering other compositions, such as small molecules, to restore cellular function and/or create permissive cellular environments for repair, regeneration, and growth.

### SUMMARY OF THE INVENTION

In some embodiments, substrates comprise a biocompatible gel and at least one mesenchymal stem cell, neural progenitor cell, or genetically modified fibroblast. One suitable biocompatible gel is alginate. Substrates contain therapeutic agents that may take the form of cells, proteinaceous and non-proteinaceous compositions released or excreted from cells of the substrate, and proteinaceous and non-proteinaceous compositions present in the substrate independent or not derived from cells in the substrate.

Certain of these fibroblasts produce therapeutic agents, such as neurotrophic factors like BDNF or NT-3.

Some substrates also contain as therapeutic agents enzymes, such as chondroitinase ABC.

In some embodiments, the enzymes are chemically modified. Modifications may include attaching a spacer, such as dextran or heterobifunctional polyethylene glycol, to the enzyme.

Substrates can be of any shape and size suitable for implant for the purpose of repairing damaged nervous tissue or restoring function to damaged nervous tissue. In some embodiments, substrates are fibrous in nature. Certain of these fibers have an average diameter of about 50-450 μm, but could extend up to about 1 mm in diameter. In other embodiments, substrates are in the form of micro or nano capsules. Certain of these capsules are with an average diameter of about 0.1-1000 μm. Other substrates can be in the form of a block with or without grooves. The block can be a disc, square, rectangular, or any other suitable shape. The grooves can be square, rounded, triangular or any other suitable shape.

Some substrates have an additional coating, such as polycations (e.g., poly-L-ornithine (PLO) or poly-L-lysine (PLL)), laminins, or portions thereof, such as the neurite outgrowth-promoting domain of laminin-111. The additional coating is not limited to macromolecules, but may be composed of small molecule compounds.

Certain substrates contain a sharp instrument, such as a suture needle or a tube, flush or pointed, which is partly embedded in one of the ends of the substrate. The sharp instrument may allow penetration of scar tissue and/or act as a guidance channel for therapeutic agents to pass into the damaged tissue or area in need of repair, regeneration, or growth.

Substrates may contain therapeutic agents, such as cells, peptides, proteins, enzymes, or compounds, that can be entrapped within or seeded on the substrate, such that they are present in a concentration gradient across the substrate. The therapeutic agents may be present in the substrate in the form of microcapsules or microspheres that may facilitate controlled and/or long-term release of therapeutic agents, especially non-cellular therapeutic agents, found therein.

Substrates may have one or more gradients of therapeutic agents, where the concentration of therapeutic agent is higher at one end of the substrate than at the other end of the substrate. For example, at a first end of the substrate, cells might be in a higher concentration than at a second end of the substrate, while enzymes are in a higher concentration at the second end of the substrate than at the first end of the substrate. In another example encapsulated or stabilized bioactive compounds could be at a higher concentration at the first end. Any combination of gradients of therapeutic agents may be present in the substrate.

In some embodiments, the substrate can have two or more regions that comprise different compositions. For example, a substrate can have a first region comprising alginate and a second region comprising neurotrophic factor producing fibroblasts encapsulated in alginate. In some embodiments, articles can be seeded with cells such as stem cells. For example, the preceding article can have a second region having grooves which are seeded with neural stem cells, which can be useful for guiding axon growth. Likewise, the seeded stem cells can be useful as cells to replace damaged or lost cells within the nervous tissue.

In certain embodiments substrates may have at least one surface comprising a plurality of grooves, said substrate comprising a biocompatible gel and at least one of a plurality of cells.

Other embodiments concern methods of molding a solution comprising filling a mold with a solution of biocompatible gel and at least one mesenchymal stem cell, neural progenitor cell or genetically modified fibroblast, and a cross-linking agent to produce a shaped body. The shaped body has any number of indentations such as grooves or circular pits. When the biocompatible gel comprises alginate, the cross-linking is accomplished by exposing alginate to calcium salts, such as calcium chloride, calcium carbonate, or calcium sulfate. Other suitable cross-linking agents include aluminum and barium cross-linking agents. In some cases the cross linking agent can be slowly released into the gel for example from a calcium carbonate salt in contact with D-(+)-gluconic acid δ-lactone. In some embodiments a cross-linking agent is absent from the solution, in which case the molding occurs by lowering the temperature of the solution.

In some embodiments the method of molding the solution is performed in succession to create a shaped body having different components of therapeutic agents, such as cells, peptides, proteins, enzymes, and/or compounds. The substrate comprises a gel formed by forming a first solution comprising alginate and a plurality of fibroblasts, producing at least one neurotrophic growth factor; and combining said first solution into a second solution comprising a calcium or other suitable multivalent salt to form said alginate gel. In another embodiment, the substrate is formed by combining an alginate solution containing an enzyme or stabilized enzyme aggregates with a second solution comprising a calcium salt to form said alginate gel. Likewise the gel can be formed with various cells, including mesenchymal stem cell, neural progenitor cell or genetically modified fibroblast.

Certain embodiments relate to substrates comprising a plurality of fibers, each fiber independently comprises a biocompatible gel and at least one mesenchymal stem cell, neural progenitor cell or genetically modified fibroblast, wherein each fiber is optionally, and independently, coated.

Some embodiments promote the differentiation of neural progenitor cells (NPCs) into neurons or glia. In certain embodiments, substrates promote differentiation of NPCs into predominantly neurons. Certain embodiments relate to substrates promote differentiation of NPCs predominantly to astrocytes. Some embodiments relate to substrates that promote differentiation of NPCs predominantly to oligodendrocytes. Embodiments may relate to substrates that promote differentiation of NPCs to predominantly Schwann cells.

Embodiments also relate to methods of repairing damaged nervous tissue or restoring the function of damaged nervous tissue by placing a substrate within the damaged area or in proximity the damaged area, such as about 0.001 mm, about 0.01 mm, about 0.1 mm, about 1 mm, about 5 mm, about 10 mm, about 25 mm, about 50 mm, about 75 mm, about 100 mm, about 150 mm, or about 200 mm away from the damaged tissue. Substrates useful for this purpose comprise a biocompatible gel and at least one mesenchymal stem cell, neural progenitor cell, or genetically modified fibroblast. The damaged nervous tissue is found in the spinal cord, brain, or peripheral nervous system. In some embodiments the damage results in the loss of cells, including neurons, astrocytes, oligodendrocytes, and Schwann cells, in which case the embodiment relates to replacing the loss cells. Certain embodiments relate to regenerating axons in the damaged tissue. Further embodiments relate to restoring myelination of neuronal processes.

Both the methods of repairing damaged tissue and restoring the function of damaged tissue relate to functional changes in the affected animal. In some embodiments, the damaged tissue occurs in the spinal cord such that the animal experiences deficits in the ability to move muscles of the periphery or sense external stimuli, in which case embodiments restore, at least partially, movement and/or sensory abilities. In other embodiments, the damaged tissue occurs in the brain such that the subject experiences deficits in cognitive function, in which case embodiments restore, at least partially, those cognitive functions. Certain embodiments are directed to damaged tissue that manifests in combination of movement, sensory, and cognitive functions, in which case embodiments restore, at least partially, the movement, sensory, and cognitive functional deficits.

### BRIEF DESCRIPTION OF THE DRAWINGS

FIG. 1 shows an example of a mold of the instant invention.

FIG. 2 shows an alginate substrate having a first region comprising alginate and a second region comprising a gradient of BDNF or NT3 producing fibroblasts encapsulated in alginate.

FIG. 3 shows an alginate substrate having a first region comprising alginate and a second region comprising BDNF or NT3 producing fibroblasts encapsulated in alginate, the second region having grooves which are seeded with neural stem cells.

FIG. 4 contains a drawing of the substrate and the viable of cells within the substrate. FIG. 4A shows an alginate substrate of the disc configuration containing genetically-modified fibroblasts (blue) encapsulated within the body of the substrate which continually delivering neurotrophic factors (pink). NPCs can be seeded on the surface of the substrate, which permits differentiation as well as provides structural support to the NPCs. FIG. 4B represents the cell viability of NPCs on various substrates coated with laminin 111 and encapsulated neurotrophic factor-producing fibroblasts evaluated by calculating the percentage of live and dead differentiated NPCs seeded on alginate, agarose, or gellan gum (n=10).

FIG. 5 contains graphs and micrographs representing the viability, morphology, and migration of NPCs using various substrates. FIG. 5A is a graph representing the NPC attachment on alginate substrates of various compositions. FIG. 5B are micrographs of NPC attachment to various geometries of laminin 111 coated alginate substrates in the shape of strings and discs after 1, 3, and 5 days in culture, scale bar=100 μm. FIG. 5C is a graph representing the NPC migration on different alginate substrates in the shape of discs. All quantification was done after NPCs had attached and differentiated for 5 days in culture (n=10). FIG. 5D are fluorescence micrographs showing the expression of neurotrophic factor receptors on NPCs seeded on laminin 111 coated alginate discs after 1 day in culture stained with the following antibodies: anti-p75 (red) anti-Trk B (green), and anti-Trk C (blue), scale bar=100 μm.

FIG. 6 are micrographs and graphs representing the multi-lineage differentiation of NPCs on various alginate substrates. FIGS. 6A and 6B are fluorescence and bright-field micrographs, respectively, showing multi-lineage differentiation of NPCs on laminin 111 and PLO coated alginate discs with or without encapsulated Fb/NT3 or Fb/BDNF. After 5 days in culture, cells were stained with the following antibodies: anti-β-tubulin (red, marking neurons), anti-MBP (green, marking oligodendrocytes), and anti-GFAP (blue, marking astrocytes), scale bar=100 μm. FIG. 6C are graphs representing the quantification of NPC differentiation profiles on alginate constructs after 5 days in culture (n=10).

FIG. 7 are micrographs showing the results of alginate substrate implantation in vivo. FIG. 7A are photomicrographs (low and high power, inset) of the recovery of the implanted alginate substrates (Laminin+PLO coat) with encapsulated Fb/BDNF after 7 days, note the distinct border of the substrate (black arrows) and presence of encapsulated fibroblasts (black arrowheads). Fibroblasts were still viable and producing BDNF after implantation on the surface of the mouse brain as seen by β-galactosidase staining. Alginate constructs without encapsulated fibroblasts did not show any positive β-galactosidase staining. FIG. 7B are fluorescence micrographs depicting negative TUNEL staining in the cortex after 7 days of implantation. FIG. 7C are micrographs of BDNF staining in the cortex after 7 days of implantation. Note the presence of high BDNF immunoreactivity in cortex in which BDNF encapsulated fibroblasts were implanted. All scale bars=100 μm.

FIG. 8 contains micrographs and a graph depicting the reduction in the severity of injury after in vivo transplantation of the alginate construct. FIG. 8A are fluorescence micrographs depicting TUNEL staining in the injury induced cortex of mice after 3 days of implantation of a sham, fibroblast free alginate construct, and the alginate construct with encapsulated Fb/BDNF. Alginate constructs with encapsulated Fb/BDNF showed the lowest amount of positive TUNEL staining Scale bar=100 μm. FIG. 8B is a graph representing the quantification of damaged cells after 3 days of implantation of alginate constructs in the cortex of a brain injury induced mice (n=6).

FIG. 9 is a photomicrograph seeded colony of NPCs. Migration distance of NPCs was calculated as a ratio of outer migration distance to the radius of the original NPC colony, scale bar=100 μm

FIG. 10 contain micrographs depicting the stem cell-like characteristics of the NPCs. FIG. 10A shows the NPCs' spherical colonies. FIG. 10B shows the NPCs dissociated into single cells. FIG. 10C shows the reformation of the cells into spherical colonies after 7 days.

### DETAILED DESCRIPTION OF ILLUSTRATIVE EMBODIMENTS

In some aspects, the invention concerns substrates comprising a biocompatible gel and at least one of a plurality of cells. Substrates contain therapeutic agents that may take several forms. Therapeutic agents may be (a) cellular in nature, meaning cells within the substrate provide a therapeutic effect; (b) “cellularly-derived,” meaning that the cells act as source of therapeutic agent by producing, excreting, or secreting a proteinaceous or non-proteinaceous compounds, or (c) “non-cellular,” meaning that the substrate contains proteinaceous or non-proteinaceous compounds that are not derived from cells of the substrate.

To protect modified cells used in the repair of nervous tissue and also to avoid the need for immune suppression, the substrate can be coated to form a semi-permeable membrane through which only beneficial species, such as therapeutic agents, can pass.

In some embodiments, cells that have been genetically modified to produce proteins or peptides, such as brain derived neurotrophic factor (BDNF) or NT-3 (neurotrophic factor 3) have been obtained. Liu, et al., Neuroreport 9, 1075-1079 (1998). These cells can be encapsulated in a gel (alginate substrate, for example), which will then be coated with large molecular weight, positively charged molecules in order to provide a semi-permeable membrane. Further a growth permissive coating can be added to the substrate. These steps ensure the release of the protein or peptide, protect the encapsulated cells from the host immune system, and also provide a growth-permissive environment for neuronal regeneration.

The substrate, unlike a collection of microcapsules (see, Tobias, et al., J. Neurotrauma, 18(3), 287-301(2001)), can be designed to produce a concentration gradient of therapeutic agent that will guide the growth of neurites through the damaged tissue, such as axons across the spinal cord lesion. Alginate administration to the damaged tissue has been shown to reduce scar formation. Incorporating certain enzymes into the substrate can contribute to the breakdown of scar tissue in the damaged tissue can further promote repair. In addition to reducing or eliminating scar tissue, other impediments to repair, regeneration, and growth, such as tangles or deposits; could be ameliorated using substrates containing enzymes and other therapeutic agents. This combination of strategies shows promise for the regeneration and guidance of neurites through the damaged tissue, which promotes recovery after the injury.

Implantation of neural progenitor cells as well as fibroblasts that produce neurotrophic factors have been shown to promote neuronal regeneration as well as neural growth. An alginate substrate, was developed to allow for the diffusion of neurotrophic factors to the injury while protecting the fibroblasts from an immune response. For immediate neuronal growth, this substrate was also seeded—any process that allows cells to grow on the substrate—with undifferentiated cortex derived neural progenitor cells (NPCs) grown as neurospheres isolated from embryonic day 14 mice and migration distance from the center was measured. After 7 days, the NPCs seeded on laminin coated alginate substrates with encapsulated NT3 producing fibroblasts had completely differentiated and migrated the farthest from the center of the original neurosphere, showing neuronal growth. These results show that this substrate is a viable strategy for delivery of neurotrophic factors and implantation in the body.

Cells that have been genetically modified to produce proteins and peptides, such as growth factors BDNF or NT-3, through ex vivo gene therapy can provide a constant, localized supply of growth factor at or in proximity to the damaged site. Similarly proteins and peptides, and other therapeutic agents, that have been microencapsulated to achieve a controlled or sustained release can provide a constant or patterned localized supply. The use of the enzyme chondroitinase ABC will degrade the scar tissue present in the damaged tissue and also contribute to repair. Enzymes that have been manipulated to have increased stability by cross linking or immobilization, can also be utilized. Similarly, cells that are genetically modified to produce other therapeutic agents can be utilized.

Certain substrate formulations have the ability to promote the attachment, survival, and/or lineage differentiation various cell types, including mesenchymal stem cells, NPCs, and genetically modified fibroblast.

Other aspects of the invention relate to the ability of the substrate to direct selective differentiation of NPCs to neurons, astrocytes, oligodendrocytes, or Schwann cells are suitable for acting a scaffolds for regenerating tissue or sources of restorative cells. Substrates disclosed herein direct NPC differentiation into predominantly neurons, astrocytes, oligodendrocytes, or Schwann; and in greater proportions than previous studies, such that the population of desired cell—neuron, astrocyte, or oligodendrocyte—is present in approximately 20%, 30%, 40%, 50%, 60%, 70%, 80%, 85%, 86%, 87%, 88%, 89%, 90%, 91%, 92%, 93%, 94%, 95%, 96%, 97%, 98%, or 99% of the overall cell population in the substrate. In some embodiments it is advantageous to limit the differentiation to astrocytes, in which case certain embodiments more effectively limit the differentiation of this cell type—to approximately 20%, 15%, 14%, 13%, 12%, 11%, 10%, 9%, 8%, 7%, 6%, 5%, 4%, 3%, 2%, or 1% of the overall cell population in the substrate—than in previous studies of NPCs seeded on alginate substrates containing encapsulated neurotrophic factor-producing fibroblasts. In addition to NPCs serving as therapeutic agents, other non-cellular therapeutic agents may be delivered through embodying substrates. Using this delivery system, therapeutic agents can be tailored for different applications.

An alginate gel substrate can act as the nerve guidance channel, providing a growth-permissive surface for regenerating neurons. By encapsulating the genetically modified cells in the substrate and coating it with a perm-selective membrane, the cells can be protected from the host immune system to prevent an immune response, act as a semi-permeable barrier for the diffusion of molecules, provide a conduit for the diffusion of non-cellular therapeutic agents, and reduce immune cell infiltration of the substrate. Using semi-permeable barriers circumvents the need for immune suppression prior to or after implantation of the substrate. This combination therapy strategy is a step towards enhanced recovery of function following acute spinal cord injury or brain or peripheral nervous system repair.

An alginate gel substrate can act as a source therapeutic agents for various neurodegenerative diseases in which cells are lost or cannot function properly. The alginate gel substrate, with its cells or other therapeutic agents, may be transplanted into a subject in need of treatment in an amount effective to treat neurodegeneration. Possible causes of neurodegeneration include, but are not limited to, prolonged hypoxia, exposure to toxins (such as alcohol), infection, genetic mutation, or trauma. As used herein, the phrase “effective to treat the nervous tissue degeneration” means effective to ameliorate or minimize the clinical impairment or symptoms of the neurodegeneration. For example, where the nervous tissue degeneration is a peripheral neuropathy, the clinical impairment or symptoms of the peripheral neuropathy may be ameliorated or minimized by alleviating vasomotor symptoms, increasing deep-tendon reflexes, reducing muscle atrophy, restoring sensory function, and strengthening muscles. In other embodiments, substrate improves or restores cognitive functions, such as arousal, attention, reasoning, perception, intelligence, learning and memory, decision-making, planning, and motor coordination. Diseases, conditions, and syndromes that could benefit from various embodiments of the invention include, but are not limited to, Alexander's disease, Alper's disease, Alzheimer's disease, amyotrophic lateral sclerosis, ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff's disease, Schilder's disease, subacute combined degeneration of spinal cord secondary to pernicious anaemia, Schizophrenia, spinal cord injury, spinocerebellar ataxia (multiple types with varying characteristics), spinal muscular atrophy, Steele-Richardson-Olszewski disease, and tabes dorsalis

Cells provided may be substantially undifferentiated or predominantly differentiated into neuronal or glial cells, and therapeutic agents may be proteinaceous, such as neurotrophic factors, peptides, enzymes, hormones, and antibodies or non-proteinaceous compositions, such as neurotransmitters or neuromodulators. Therapeutic agents may be released from the provided cells; or may act within the cells to create releasable compounds, such as neurotransmitters or anti-oxidants; or act as a source of agents that require a cellular environment to function, such as particular receptors or enzymes.

The amount of cells or other therapeutic agents effective to treat nervous tissue degeneration in a subject in need of treatment will vary depending upon the particular factors of each case, including the type of neurodegeneration, the stage of the neurodegeneration, the subject's weight, the severity of the subject's condition, the type of differentiated cell required for treatment, and the method of transplantation. This amount may be readily determined by the skilled artisan, based upon known procedures, including clinical trials.

Where neuronal cells are desired, the provided cells may act to replace lost cells and/or reestablish lost circuitry. Additionally, providing astrocytes in proximity to damaged tissue can modulate levels of extracellular neurotransmitters, calcium, or reactive oxygen species, which in turn may contain the extent of damage and promote repair and restoration of the tissue's function. Likewise, substrates could be used to provide oligodendrocytes or Schwann cells such that myelination could be restored to the damaged tissue.

Neurotrophic factors are proteins that enhance neuronal proliferation, survival, differentiation, migration, axon growth, and synaptic plasticity. These neural growth factors include nerve growth factor (NGF), brain-derived neurotrophic factor (BDNF), glial cell-line derived neurotrophic factor (GDNF), ciliary neurotrophic factor (CNTF) and neurotrophin-3 (NT-3), among others. Studies using neurotrophic factors to treat SCI have shown that they promote neural repair and recovery after injury in the CNS. In vitro, neurotrophic factors have been shown to enhance axonal and dendritic growth. The use of these neurotrophic factors alone or in combination has been shown to aid in axonal regeneration in the injured spinal cord. However, systemic administration is not possible, because neurotrophic factors cannot cross the blood-brain barrier. Repeated invasive techniques would have to be employed to administer neurotrophins locally. One solution is the implantation of genetically modified cells that continually produce these neurotrophins or other compounds that cannot cross the blood-brain-barrier.

In addition to growth factors, other proteinaceous forms of therapeutic agents include cytokines, chemokines, antibodies, peptides, and glioma toxic proteins. Therapeutic agents may also include compounds known to treat nervous-system associated disorders, such as, but not limited to, levodopa, dopamine receptor agonist, such as pergolide; galantamine, rivastigmine, donepezil, tacrine, and glatiramer acetate, potential protectants against Familial Alzheimer's Disease (FAD) such as Humanin

One suitable biocompatible polymer is alginate. Alginate is a linear, water soluble polysaccharide derived from seaweed, consisting of 1,4-linked α-L-guluronic acid (G) and β-D-mannuronic acid (M) monomers. In the presence of multivalent cations, usually Ca²⁺, alginate forms gels by the interaction of these cations with blocks of guluronic acid residues. Alginate has also been shown to enhance nerve regeneration in the peripheral and central nervous systems. Coating with polycations such as poly-L-ornithine (PLO) or poly-L-lysine (PLL) creates a size exclusion barrier that restricts the passage of high molecular weight substances into and out of the gel. An additional coating of laminin or other basement membrane proteins will support cell adhesion to the alginate substrate.

Calcium, barium, and aluminum salts are among the useful alginate cross-linking agents. Such compounds include calcium chloride, calcium carbonate, and calcium sulfate. These compounds can be contacted with the alginate, for example, in the form of an aqueous solution, as solid salts such as calcium sulfate, and as slow release formulations of calcium carbonate and glucono-δ-lactone.

The alginate gels may be optimized reach and/or maintain physiological pH., between 7.2 and 7.4 Alginate gels, including calcium carbonate-GDL-alginate discs, encapsulated Fb/BDNF/NT3 and crosslinked with either calcium chloride (CaCl₂), calcium sulfate (CaSO₄), or calcium carbonate (CaCO₃ show a range of pH after 4 days of incubation in culture medium. Alginate constructs crosslinked with Ca²⁺ from calcium carbonate achieved the optimal pH for cell growth (7.3±0.2, p<0.001) after 4 days in culture. (Table 1).

However, alginate constructs crosslinked with Ca²⁺ from calcium sulfate and calcium chloride presented a more acidic cell growth environment (pH of media in CaSO₄ construct: 5.1±0.2, CaCl₂ construct: 5.0±0.1, p>0.05). This can be attributed to that fact that the alginate constructs crosslinked with Ca²⁺ from calcium carbonate contained bicarbonate ions that acted as buffering agents against toxic cell by-products within the culture media. Hence it can be concluded that calcium carbonate-GDL-alginate discs provide an extremely favorable cell growth environment for cells which favor these neutral pH values.

TABLE 1
pH of culture media on alginate constructs after 4 days, n = 6

| Alginate Construct | |
|---|---|
| (crosslinked with Ca²⁺) | pH of Media After 4 Days |
| CaSO₄ | 5.1 ± 0.2 |
| CaCl₂ | 5.0 ± 0.1 |
| CaCO₃ | 7.3 ± 0.2 |

Substrates of the present invention can be made by a variety of methods. These methods include positive mold and negative mold methods.

A mold is a scaffold which is a three dimensional representation of a desired shape of the substrate. The mold can have positive features (extending up from a device surface) and negative features (extending into the device surface). A deformable substance (for example a gel) placed on a positive mold will fill the areas between the extending features, for example producing fibers when placed in grooves, but will fill and cover grooves in the surface of a negative mold resulting in a block of gel into which grooves are imprinted.

A positive mold method can be made by many means such as for example a block of Teflon®, a synthetic fluoropolymer marketed by DuPont, into which grooves are formed by machining or a laser, photolithography using for example a positive photo resists such as phenolic resins, a machined stainless steel mold and the like. The method produces grooves of the desired size (down to submicron depending on the method of making the mask) which can be filled with hydrogel (such as alginate). The desired elements can be placed into the gel (nerve growth factor, nerve growth factor excreting cells, enzyme, immobilized enzyme, stem cells, Schwann cells, and the like) in a defined location (i.e. gradients and patterns can be created, if desired) and concentration. In addition, the resulting substrates that were formed in the grooves can be coated with growth-permissive substrates (polymer coatings, modified polymer coatings, peptides, laminin, etc). The pre-formed “fibers” once set can be extracted and used “as is” or with further processing (coating with other polymers, factors etc.). Substrates can be placed in proximity to an injured spinal cord or brain or peripheral nervous tissue.

In regard to negative molds, they are formed for example, by generating grooves in a block of hydrogel. Grooves can be produced in the block by any suitable method. These methods include pre-formed ridges of suitable shape and size in the mask, or cutting away a groove in a blank mask by a suitable means such as a knife or laser, depending on the desired size and shape, or placing items such as rods in the setting gel and removing them after setting to leave a groove of the desired size and shape. The blocks can optionally be manipulated by the addition of gradients, etc. Also post gelling modification can optionally be utilized (coating with other polymers, factors, etc.). In the case of the negative mold, the entire block can be placed in proximity to the damaged tissue. In some embodiments, the block can be formed in a shape suitable for the site of implantation. In other embodiments, the block's shape can be modified for an improved fit based on the implantation site.

In yet other embodiments, the mold (or block) can contain micro or nanofibers. The micro/nanofibers can optionally be filled with fibroblasts capable of producing neurotrophic factors or other agents that are useful in promoting neural growth.

In some embodiments, the substrate can comprise alginate, alone or in combination with other agents such as poly(ethylene oxide) (also referred to as “PEO”), in the form of nanofibers. Nanofibers can be spun by any suitable method. See, for example, Bhattarai, et al., Adv. Mater. 2006, 18, 1463-67. In some embodiments the weight ratio of alginate to PEO is 40:60 to 90:10. In some embodiments, the weight ratio is 70:30 to 80:20.

As used herein, the term “fiber” is intended to mean structure having a high ratio of length to width. Cross-sections of the fibers used herein are typically round, rectangular, or square, but can be any useful shape.

Certain substrates additionally comprising enzyme as therapeutic agents. One suitable enzyme is chondroitinase ABC, or other enzymes such as, sialidase, hyaluronidase. Therapeutic agents within the substrate may be antibodies, such as NI-35/250, other growth factors, such as glial-cell-line-derived neurotrophic factor, or hormones, such as testosterone, dihydrotestoterone, progeterone or estradiol; chemokines, or cytokines; peptides such as humanin. Therapeutic agents may be stabilized by mechanical (microencapsulation and the like) or chemical (cross linking with bifunctional agents such as gluteraldehyde and the like). For example, proteinaceous therapeutic agents, such as enzymes like chondroitinase ABC, may have spacer arms, such as dextran, attached to them.

Some substrates are coated with a growth permissive membrane and/or a rate limiting membrane. Coating with polycations such as poly-L-ornithine (PLO) or poly-L-lysine (PLL) creates a size exclusion barrier that restricts the passage of high molecular weight substances into and out of the gel. An additional coating of laminin or other basement membrane proteins will support cell adhesion to the alginate substrate and promote cell differentiation.

Some substrates have more than one coating. For example, after coating with a with a growth permissive membrane and/or a rate limiting membrane, substrates may be further coated with an enzyme-modified alginate. For example, chondroitinase ABC enzyme may be covalently attached to alginate using an adaptation of aqueous carbodiimide chemistry. Hermanson, Bioconjugate Techniques, Academic Press, San Diego, Calif. p 169-176 (1996). Further, the alginate-chondroitinase ABC conjugate can be used as the substrate itself.

In other embodiments a bundle of several fibers each containing different components, may be either tied together with e.g. suture material, or just placed parallel to each other, for example a fiber with chondroitinase ABC could be made with no rate controlling membrane so that relatively rapid release is obtained, next to a fiber with cells and a gradient and even also with more chondroitinase ABC. Likewise, all grooves in a block of alginate need not be identical—some grooves could be patterned differently than others. In the slab-groove form it could even mimic a real spinal cord or other nervous tissue. In addition, some fibers could be comprised of a different hydrogel, such as agarose.

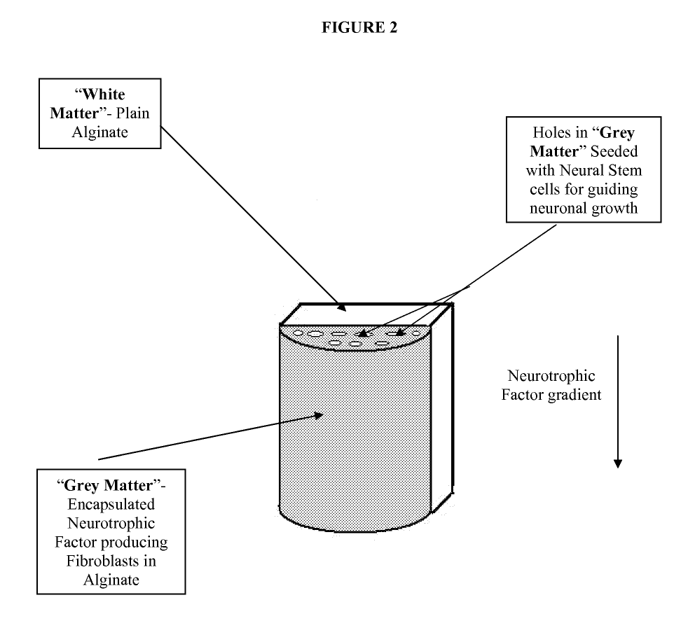

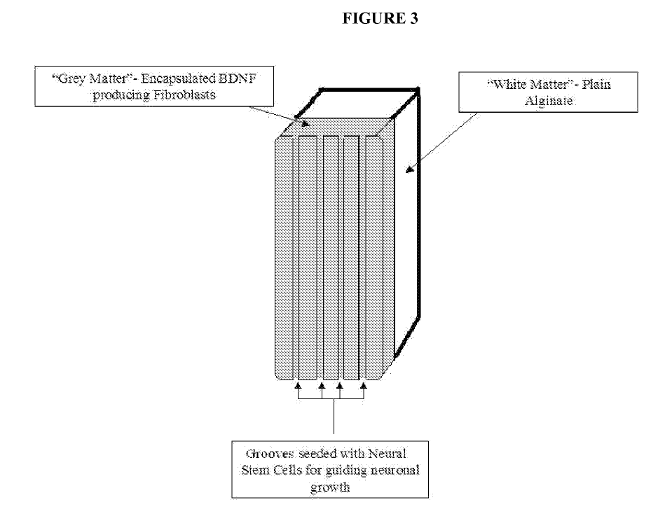

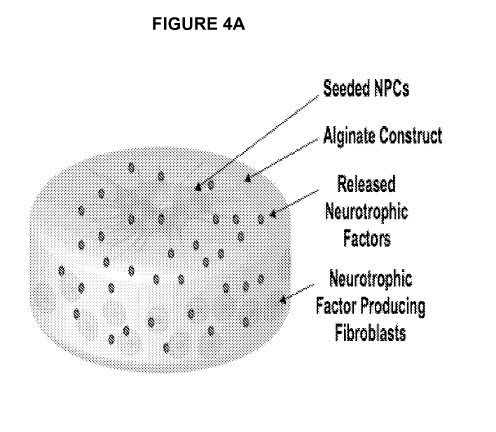

In yet other embodiments, the substrate need not be uniform in composition. Portions of the substrate may contain useful agents while other portions may contain alginate either plain or with other useful agents. For example, the portion of the article to be in contact with the spinal region may contain simulated “grey matter” (alginate encapsulated fibroblasts or other useful agents) and the region remote from the spinal region may contain simulated “white matter” (plain alginate). See, for example, FIGS. 2 and 3.

The invention is illustrated by the following examples that are not intended to be limiting.

### Examples

### Example 1

### Production of Alginate Fibers

Alginate fibers were made by the following method. Sterile-filtered alginate solution (ranging from 1% (w/v)-1.5% (w/v) containing a species of interest (genetically modified fibroblasts, enzyme, etc.) were loaded into a sterile 5 ml syringe fitted with a non-beveled 22 gauge needle, which is then mounted on a Kent Genie syringe pump, e.g., Kent Genie or Sage, which had been wiped down with 70% isopropyl alcohol and placed under UV for 15 minutes in a plasma hood. The tip of the needle was submerged in a beaker containing a 1.3% calcium chloride solution and the syringe pump flow rate is set (range between 10-100 ml/min. Calcium cross-linked alginate fibers were produced by injection of alginate into the calcium chloride solution and are left to harden in this solution for one hour. The fibers were washed with autoclaved HEPES buffer (pH 7.4).

### Example 2

### Alternate Method of Producing Alginate Substrates

A slow gelling method for creating alginate substrates consisted of forming a 5.5 g/L solution of calcium carbonate and a 28 g/L solution of D-(+)-gluconic acid δ-lactone (GDL) which were sterilized by autoclaving. A 1% (w/v) alginate solution was sterilized by filtration through a 0.45 μm sterile filter. The calcium carbonate solution was added to the alginate at a ratio of 0.5 ml CaCO₃ solution per ml of alginate. GDL solution was added to the alginate-calcium carbonate mixture to reach a final GDL concentration of 80 mM (a ratio of 0.5 ml GDL per ml of alginate). The entire mixture was shaken and poured into graft molds and allowed to incubate at 37° C. for 1 hour to harden. Substrates were washed with sterile HEPES buffer (pH 7.4) prior to coating with PLO and an isoform(s) of the laminin family.

### Example 3

### Production of Alginate Discs

Alginate discs were produced by a slow gelling method. Calcium carbonate was slowly solubilized by the gradual dissolution of D-glucono-delta lactone (GDL) which lowered the pH. Kou & Ma, Biomaterials 22, 511-21 (2001). A 1% (w/v) sterile filtered (0.45 micron bottle top filter) alginate solution was poured into a sterile 15 ml centrifuge tube. An aqueous calcium carbonate solution was added and the solution was mixed, followed by the addition of an aqueous GDL solution, creating a slurry with a final GDL concentration of 80 mM. All aqueous solutions were first sterilized by autoclave. A 2 ml aliquot of the slurry was poured into each well of a 6 well culture plate and allowed to incubate at 37° C. for 24 hours in order to harden. The resulting discs were then washed sufficiently with HEPES buffer (pH7.4) and had a characteristic surface area of 9.6 cm². Disc for in vivo use had a total characteristic surface area of 0.08 cm² and were fashioned from the larger discs by using a sterile punch of the desired size.

### Example 4

### Production of Alginate Fibers Using a Teflon Mold with Grooves

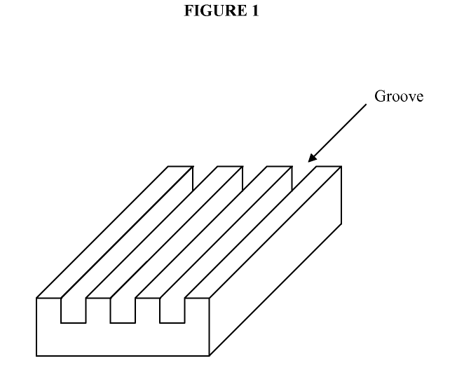

Using the novel method of slow gelling alginate as previously described in Example 2, another substrate was created (FIG. 3). The entire CaCO₃-GDL-Alginate mixture was shaken and poured into a sterile Teflon® mold (autoclaved) with grooves as indicated in FIG. 1. The substrate was then washed with sterile HEPES buffer (pH 7.4) prior to coating with PLO and laminin. The fibers were coating with sterile PLO and laminin.

### Example 5

### Coating Alginate Substrates

Alginate substrates were coated with a compound, such as PLO having a molecular weight of 15,000-30,000 at 0.5 mg/ml of alginate for 6 minutes. The PLO solution used was 6 times the volume of the alginate used for making the fibers or discs. The PLO solution was prepared in HEPES buffer just before its addition and was filtered using a sterile 0.2 μm cellulose acetate filter. The fibers and discs were then washed sufficiently with HEPES buffer to remove any unincorporated PLO.

Alginate substrates were coated with another compound, such as with laminin 111 or peptide-modified alginate ((YIGSR), Tyrosine-Isoleucine-Glycine-Serine-Arginine, attached to alginate by a carbodimide reaction. In the case of laminin 111, fibers and discs were exposed to 1 ml of a sterile laminin 111 (25%) solution for 24 hours. Finally the fibers and discs were washed with sterile HEPES buffer to remove excess unreacted laminin 111. The resulting fibers are about 350-450 μm in thickness.

### Example 6

### Enzyme Attachment Using a Spacer

Chondroitinase ABC enzyme was covalently attached to the alginate using a heterobifunctional polyethylene glycol (PEG) spacer arm containing NHS ester and maleimide functional end groups. Alginate was first thiolated through modification of a method described by Bernkop-Schnürch et al., J. Controlled Release 71, 277-285, 2001. Carboxylic acid groups were activated by adding 1-ethyl-3-(3-dimethylaminopropyl) carbodiimide (EDC) (the amount of EDC varied based on the percentage of carboxyl activation needed) to a 1% alginate solution and stirring for 45 minutes at room temperature. L-cysteine monohydrate hydrochloride was added to the solution in a weight-ratio based on the percentage thiolation desired and the pH was adjusted to 4.0. The mixture was stirred for 2 hours at room temperature before raising the pH to 6.0 and stirring for an additional hour. The thiolated alginate was then dialyzed once (3500 MWCO) at 4° C. against 1 mM HCl, followed by dialysis twice against a solution of 1 mM HCl and 1% NaCl, and then against 1 mM HCl. The thiolated alginate was lyophilized and stored at −20° C. until further use.

To attach Chondroitinase ABC to the thiolated alginate, chondroitinase was dissolved in PBS buffer (pH 7.2) and NHS-PEG-Maleimide was added to the solution in a 10-fold molar excess. The reaction mixture was incubated while stirring for 2 hours at 4° C. Excess PEG was removed using a desalting column equilibrated with PBS. Reconstituted thiolated alginate and chondroitinase-PEG were mixed in a molar ratio corresponding to that desired for the final conjugate and this mixture was incubated while stirring for 2 hours at 4° C. The mixture was then lyophilized and stored at −20° C. until further use.

### Example 7

### Cultures of Genetically Modified Fibroblasts

Fibroblasts from adult Sprague-Dawley rats that have been genetically modified with a recombinant retrovirus to release BDNF (FB/BDNF) or neurotrophin-3 (FB/NT-3) were obtained from Dr. Itzhak Fischer at the Drexel University College of Medicine. See, Liu, et al., Neuroreport 9, 1075-1079 (1998). The retroviral vector contained the human BDNF or NT-3 transgene and the reporter gene LacZ, which codes for the bacterial enzyme β-galactosidase. Fibroblasts were cultured in 10 cm tissue culture plates in Dulbecco's Modified Eagle Medium (DMEM) with 10% fetal bovine serum (FBS) and 1% antibiotic/antimycotic solution, such as penicillin/streptomycin at 37° C. and 5% CO₂. The cells are passaged using 0.25% trypsin-EDTA to approximately 70-80% confluency.

### Example 8

### Cultures of Neural Progenitors Cells

Neural progenitor cells (NPCs) were derived from the telencephalon of embryonic day 14 mice and cultured DMEM/Ham's F-12 (50:50, Gibco) supplemented with 2% B-27 (Gibco), 20 ng/ml bFGF (basic fibroblast growth factor, Invitrogen) and 20 ng/ml EGF (epidermal growth factor, Invitrogen) at 37° C. and 5% CO2. NPCs were cultured as neurospheres and passaged at 7 days after the neurospheres were visibly spherical using 0.25% trypsin EDTA.

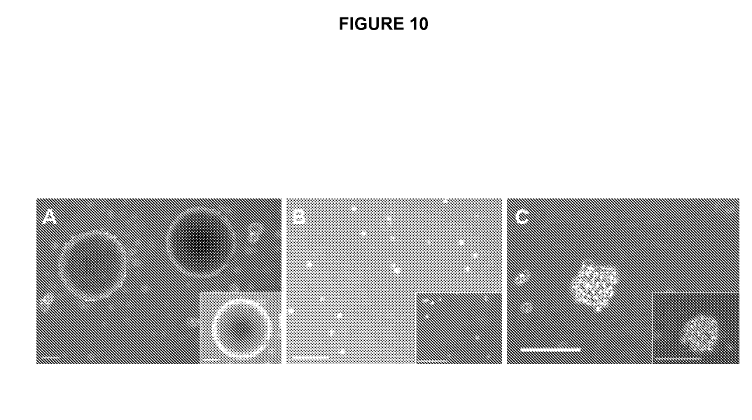

The stem cell-like properties of the NPCs was confirmed. NPCs were grown as spherical colonies (FIG. 10A), dissociated into single cells (FIG. 10B), and reformed into spherical colonies after 7 days (FIG. 10C).

### Example 9

### Incorporating Genetically Modified Fibroblasts into Alginate Substrates

Fibroblasts expressing either BDNF or NT-3 (FB/BDNF or FB/NT-3) were harvested at about 70-80% confluency with 0.25% (w/v) trypsin and resuspended in 0.5 ml sterile HEPES buffer. The cells, suspended in sterile HEPES, were added to the filter-sterilized (0.45 μm) alginate solution to obtain a 1% (w/v) alginate solution with an encapsulated cell concentration of about 3×10⁶ cells/ml of alginate solution. Alginate fibers and discs containing the Fb/BDNF or Fb/NT3 were prepared and coated as outlined in Examples 1 and 3, respectively. Fibroblast growth medium was added to the alginate substrates and incubated at 37° C. Alginate substrates containing FB/BDNF or FB/NT-3 were prepared and coated with PLO and laminin as previously described in Example 5.

FB/BDNF and FB/NT-3 were also seeded on alginate discs in such an arrangement as to form a concentration gradient of growth factor.

To determine whether the encapsulated or seeded cells continue to excrete growth factors in culture was determined by measuring the expression of the Lac-Z reporter gene, using X-gal staining as described in Example 11 below.

### Example 10

### Incorporating Neural Progenitor Cells into Alginate Substrates

NPCs were seeded on the surface of the alginate scaffold coated with laminin 111 as previously described. Seeding of the NPCs was accomplished by gently pipetting them onto the surface of the scaffold with a final cell concentration of the NPCs on the scaffold of 200,000 cells/ml of alginate used.

### Example 11

### Ability of Alginate Substrates to Support Viability, Attachment, and Initiate Lineage Differentiation of Neural Progenitor Cells

Substrates made of alginate, gellan gum, and agarose were formed into the shape of discs and coated with laminin 111 (a heterotrimeric extracellular matrix (ECM) protein). The viability of NPCs in the substrate was assessed using the Live/Dead Reduced Biohazard Viability/Cytotoxicity Kit #1(Invitrogen), cells were incubated for 15 minutes at room temperature with Component A and Component B. Cells were then fixed in 4% glutaraldehyde (Sigma, St. Louis, Mo.) for 1 hour at room temperature. Images were acquired using an Olympus IX71 fluorescence microscope. Images were processed using SPOT image software to adjust intensity levels. Images of stained cells in 10 adjacent fields were then counted blindly for each marker.

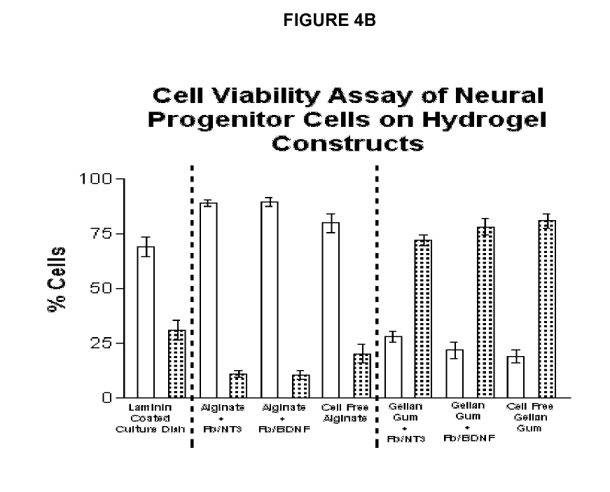

The viability of NPC's in alginate, gellan gum, and agarose was determined as shown in FIG. 4B. Alginate substrates with and without encapsulated neurotrophic factor producing fibroblasts (both Fb/BDNF and Fb/NT3) showed the highest NPC survival after 7 days in culture (cells alive on fibroblast free construct: 79.8±9.3%; Fb/NT3 construct: 89.0±3.1%; Fb/BDNF construct: 89.6±4.6%;), significantly more than control laminin 111 coated culture dishes (cells alive: 69.0±10.5%; p<0.001). Gellan gum constructs failed to provide a favorable cell growth environment and a considerably high amount of cells did not survive after 7 days in culture on the scaffold (cells alive on Fb/NT3 construct: 28.2±5.6%; Fb/BDNF construct: 21.9±8.5%). Agarose constructs did not promote any cell attachment, and hence cell viability tests could not be performed or quantified.

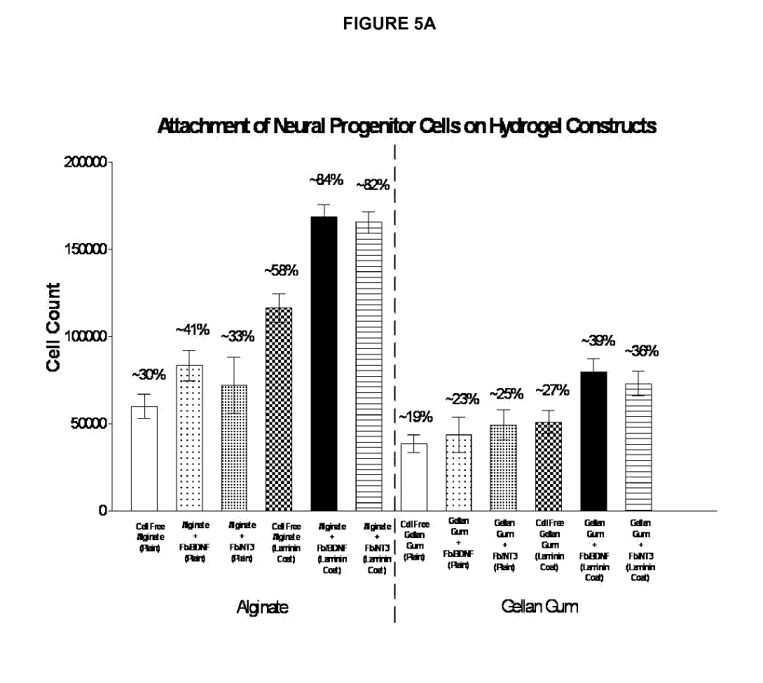

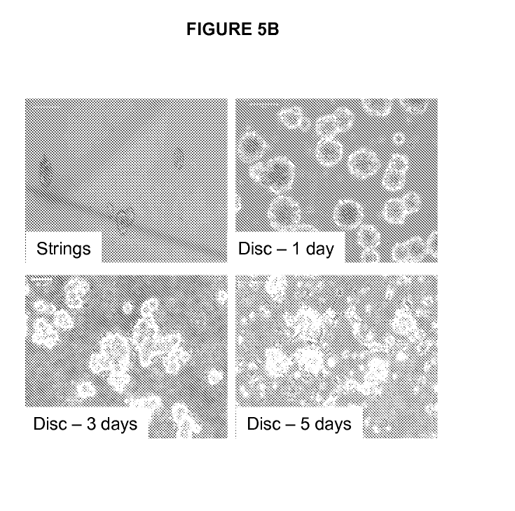

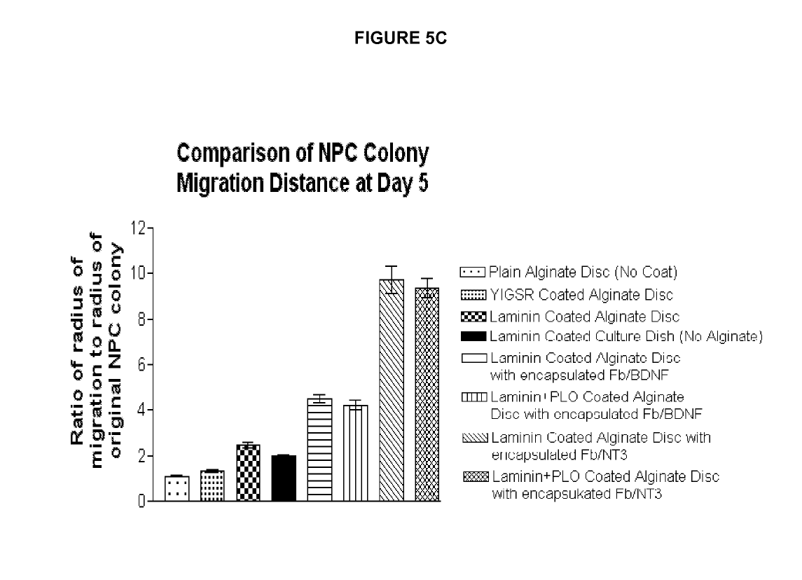

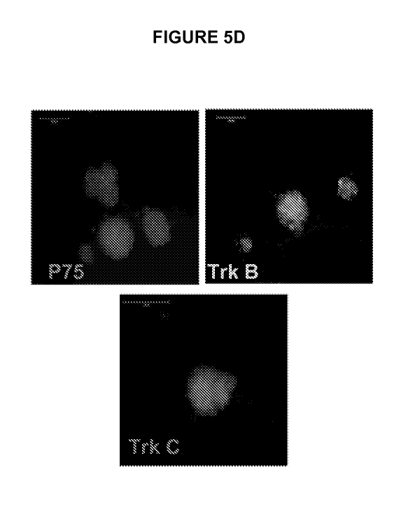

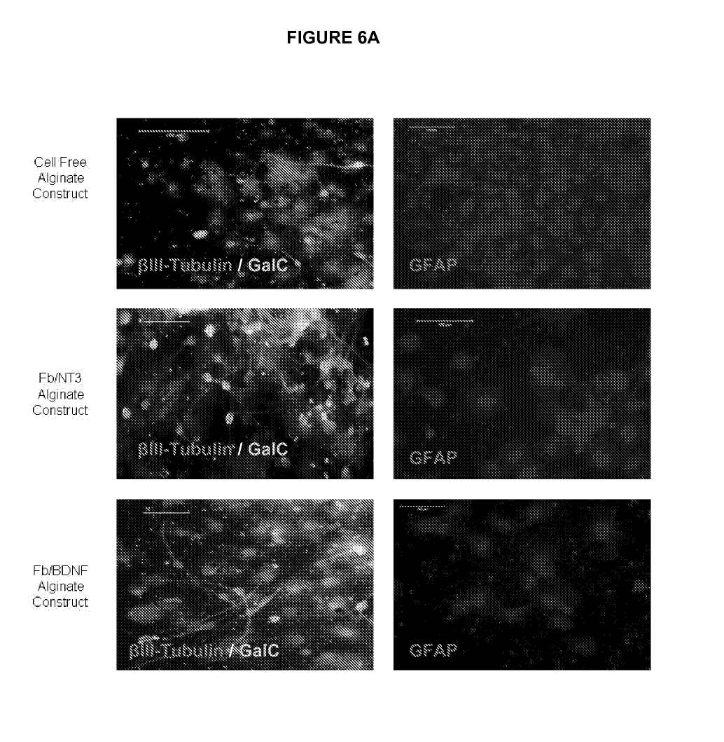

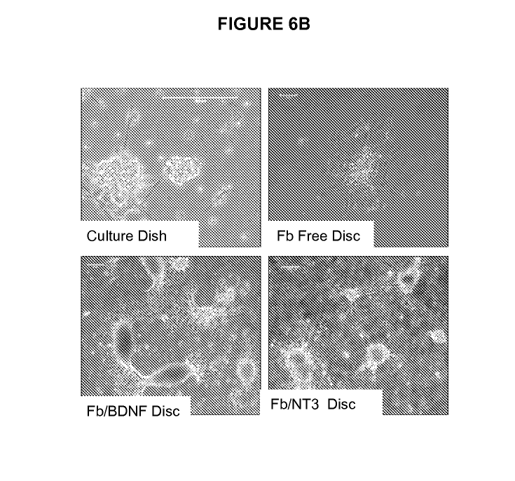

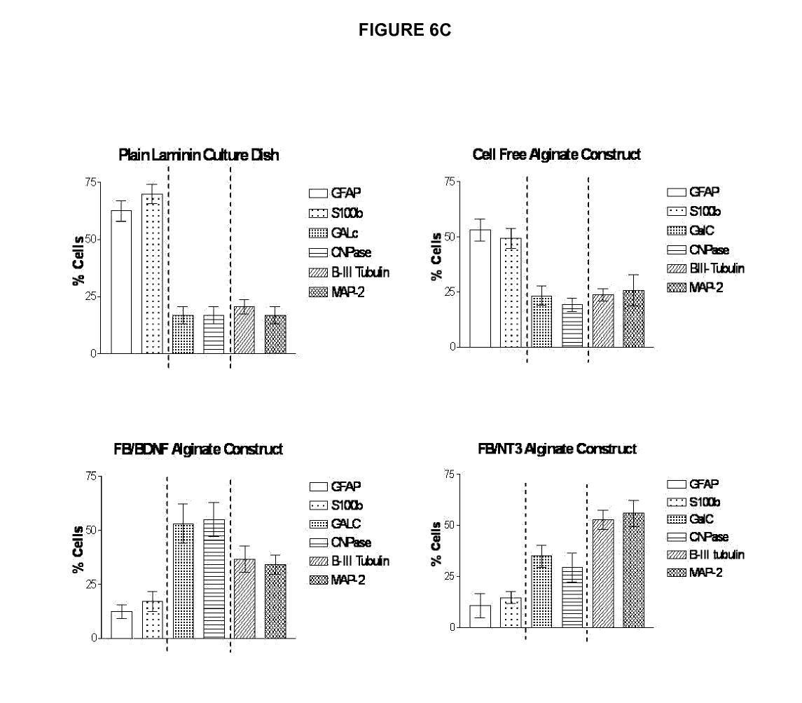

Given the effectiveness of alginate substrate to promote NPC viability, the shape of the substrate and the laminin 111 coating were assessed for supporting attachment and viability. Approximately 200,000 cells were seeded on both plain and laminin 111-coated alginate, gellan gum, and agarose constructs in the form of flat discs (FIG. 5A). Fb/BDNF and Fb/NT3 were encapsulated in some of the constructs as well. After just 1 day in culture, NPCS showed attachment to the alginate constructs with a laminin 111 coat (˜58%) while very few cells attached to alginate constructs without a laminin 111 coating (˜30%) (FIGS. 5A and 5B) (p<0.01). The addition of neurotrophic factor producing fibroblasts seemed to enhance NPC attachment as well (Plain alginate construct+Fb/BDNF: ˜41%; plain alginate construct+Fb/NT3: ˜33%; laminin 111 coated alginate construct+Fb/BDNF: ˜84%; laminin 111 coated alginate construct+Fb/NT3: ˜82%). NPCs attached to the flat laminin coated alginate constructs showed differentiation and cell migration out of the original colonies (FIG. 5B). Gellan gum constructs hindered NPC attachment (plain gellan gum construct: ˜19%; laminin 111 coated gellan gum construct: ˜27%), although the addition of neurotrophic factors did increase attachment significantly (p<0.01). Similar attachment was observed when neurotrophin producing fibroblasts (BDNF and NT3) were encapsulated within the alginate constructs (FIG. 6B).

The migratory propensity of NPCs in the alginate substrate was assessed. NPCs were seeded on different substrates and migration during a 5 day period was quantified by measuring the radius of the original NPC colony and measuring the radius of migration distance of the cells out of the original colony (FIG. 5C). These two values were compared as a ratio (Migrated Radius: Original Radius) to normalize all NPC colony sizes. 10 adjacent fields were quantified for migration distance for each type of substrate. Alginate substrates as well as substrates comprised of alginate chemically modified with the peptide YIGSR (a laminin binding motif) promoted minimal migration of NPCs, with the average ratio of migration distance to the original neurosphere radius being 1.1±0.1 and 1.3±0.1, respectively (p>0.05). However, laminin 111-coated alginate substrates and laminin 111-coated culture dishes promoted more extensive NPC migration (2.5±0.3 and 2.0±0.2 times the distance of the original radius, respectively; p<0.05). The incorporation of Fb/BDNF or Fb/NT3 within the alginate substrate increased migration distance of NPCs to 4.5±0.6 and 9.8±1.9 times the original radius, respectively (p<0.01). The addition of a poly-L-ornithine (PLO) coating to the alginate substrates with encapsulated Fb/BDNF and Fb/NT3 did not significantly change affect migration distance (p>0.05).

The ability of neurotrophic factors secreted by the encapsulated fibroblasts to interact with NPCs was determined by assessing the expression of neurotrophic receptors by NPCs. Staining for p75 showed that 79.5±2.1% of NPCs stained for the p75 receptor (FIG. 5D). 67.2±3.6% of NPCs cells stained for TrkB and 65.8±1.9% for TrkC receptors. Hence, most NPCs express at least one neurotrophic factor receptor and can bind the released neurotophic factors (BDNF and/or NT-3).

As NPCs have a multi-lineage differentiation potential (neurons, oligodendrocytes, and astrocytes), immunohistochemical analysis was used to determine the phenotypes of the cells differentiated from NPCs. After 5 days in culture, cells derived from NPCs seeded on alginate constructs with or without encapsulated neurotrophic factor-producing fibroblasts were immunoreactive with antibodies against βIII-tubulin, MAP-2, GalC, CNPase, GFAP, and S100b indicating that the alginate constructs allowed NPCs to differentiate into all three distinct cell lineages (FIG. 6A).

For immunostaining, cells were fixed in 2% PFA (paraformaldehyde, Sigma, St. Louis, Mo.) for 30 minutes at room temperature. Fixed cells were blocked for a minimum of 30 min in PBS containing 0.1% Triton X-100 (Amresco) and 10% normal goat serum (Sigma) to block non-specific antibody binding, followed by incubation overnight with the relevant primary antibodies at 4° C. After the samples were incubated with the appropriate secondary antibodies, DAPI counter-staining was used when required, and images were acquired using an Olympus IX71 fluorescence microscope. Images were processed using SPOT image software to adjust intensity levels. The following antibodies (Stem Cell Technologies Inc) were used for immunohistochemistry: anti-neuronal class III β tubulin (mouse IgG2a,1:1000), anti-GFAP (rabbit polyclonal, 1:200), and anti-MBP (rabbit polyclonal, 1:200). Appropriate Alexa Fluor 488 and Alexa Fluor 568—conjugated IgG (1:100-1:500, Invitrogen) were used as secondary antibodies. Images of stained cells in 10 adjacent fields were then counted blindly for markers for each phenotype.

NPCs exhibited different patterns of cell morphology and migration when seeded on the three different alginate constructs or grown on a plain laminin 111-coated culture dishes, exhibited different patterns of cell morphology and migration (FIGS. 5B and 5C). NPCs seeded in the laminin 111-coated culture dishes retained their NPC morphology. Cells seeded on laminin 111- and PLO-coated alginate constructs without any encapsulated fibroblasts, kept their spherical form and migrated with an even distribution around the original colony. Cells seeded on laminin 111 and PLO-coated alginate constructs with encapsulated Fb/BDNF lost their round shape and formed into oval spheroids while migrating out in a curved pattern. NPCs seeded on laminin 111- and PLO-coated alginate constructs with Fb/NT-3 kept their round shape while differentiated cells did not migrate out in an even area, but rather projected outwards as straight fasculations (FIG. 6B).

To quantify the differentiation of NPCs on the different alginate constructs, the percentages of cells that were positively differentiated into neurons (βIII-tubulin, MAP-2), oligodendrocytes (GalC, CNPase), and astrocytes (GFAP, S100b) were determined (FIG. 6C). Of the NPCs seeded in culture dishes and on alginate constructs without encapsulated fibroblasts 62.5±4.5% and 53.0±5.1% were GFAP positive, respectively. Additionally, 69.9±4.1% of the NPCs seeded in laminin-111 coated cultured dishes and 49.2±4.4% of the cells seeded on plain laminin-111 coated alginate constructs were S100b positive. Both were markers for astrocytic differentiation. The differentiation patterns for both these cases were significantly different in that the plain laminin 111 coated alginate constructs showed a significantly lower astrocyte production and different percentages of cells differentiated towards each lineage (p<0.01). The alginate construct itself seemed to be reducing astrocytic differentiation. However, 53.1±9.1% (GalC positive) of the cells seeded on alginate constructs with encapsulated Fb/BDNF and 54.9±8.0% (CNPase positive) of the cells exhibited an oligodendrocyte phenotype. Conversely, only 35.0±5.4% and 29.3±7.2% of the cells seeded on alginate constructs with Fb/NT-3 expressed GalC and CNPase respectively, whereas 52.7±4.7% (βIII-tubulin positive) and 55.9±6.5% (MAP-2 positive) of the cells had differentiated into neurons. Both types of constructs with encapsulated fibroblasts showed a significantly lower astrocyte production [12.3±3.3% (GFAP positive); 17.1±4.5% (S100b positive) for Fb/BDNF and 10.79±5.9% (GFAP positive); 14.7±2.9% (S100b positive) for Fb/NT-3] as compared to those cells seeded in culture dishes or on cell-free alginate constructs (p<0.01). Additionally, there was no statistical significance between different markers for the same lineage for each case tested (p>0.05). Hence, lineage directed differentiation of NPCs is possible through choice of the alginate construct employed.

The expression of neurotrophic receptors on the NPCs seeding in alginate discs was assessed by immunostaining as described above. Anti-p75 (mouse IgG2a, 1:100), anti-Trk B (mouse IgG2a, 1:100), anti-Trk C (mouse IgG2a, 1:100) were obtained from (Stem Cell Technologies Inc). Staining for p75 showed that 79.5±2.1% of NPCs stained for the p75 receptor (FIG. 5D). 67.2±3.6% of NPCs cells stained for TrkB and 65.8±1.9% for TrkC receptors.

### Example 12

### In vivo Transplantation of Alginate Substrates

C57BL/6 male mice were purchased from The Jackson Laboratory (Bar Harbor, Me.). Mice were anesthetized using the inhalation anesthetic Isoflurane (2½-5%) mixed with oxygen. Animals were maintained under anesthesia throughout the procedure. The level of anesthetic was assessed by monitoring respiration (>20/min), corneal reflex (air puff to eye) and leg jerk in response to pressure on the tail or hind paw. The areas of incision (scalp) was shaved with a #40 clipper blade and swabbed with 70% alcohol and betadine solution. All surgical instruments were autoclaved prior to use in a hot bead sterilizer. A sagittal incision was made in the scalp, and the skull exposed. A 3-5 mm hole was drilled in the scalp with a dremmel drill, after which, the alginate discs, with and without encapsulated FB/BDNF was placed on the brain parenchyma. Following surgery, the skull hole was packed with gel foam and the skin overlying the skull was sutured shut. The animals were placed on a surgical water heating pad during recovery from anesthesia and monitored every hour for 10 hours for recovery. To track mitotic cells in embryos, mice were injected with 500 mg/kg body weight BrdU (Sigma) one a day for 4 days.

For histological analysis, adult brains were retrieved after a paraformaldehyde (PFA) perfusion and then fixed in 4% PFA in PBS overnight at 4° C. before being transferred to sequential 20% and 30% solutions of sucrose (w/v) and left at 4° C. overnight or until the brains equilibrated. The brains were then embedded in TissueTek (Sakura) prior to cryostat sectioning (7 μm, Leica CM3050S). Sections were counter-stained with 4′,6-Diamidino-2-phenylindole dihydrochloride (Dapi, Sigma) in order to visualize the DNA and images were acquired using an Olympus IX50 fluorescence microscope. Images were processed and adjusted using MagnaFire and Photoshop 6.0 (Adobe) to include the entire signal within the dynamic range. For cell death analysis, a TUNEL (terminal deoxynucleotidyl transferase biotin-dUTP nick end labeling) assay kit (Millipore) was used. For BDNF staining, the sections were first quenched with 0.3% hydrogen peroxide for 30 min and then treated for 20 min with a blocking buffer containing PBS with 5% normal goat serum (Sigma) and 0.3% triton X-100 (Sigma). Sections were then incubated overnight at 4° C. with an anti-mouse IgG2a BDNF primary antibody (Santa Cruz, sc8042) at a 1:200 dilution in blocking solution. The sections were then washed sufficiently with PBS and exposed to a biotinylated anti-mouse secondary antibody (Vector Labs BA-2000) at a 1:100 dilution in blocking solution for 90 min at 25° C. After sufficient washing with PBS, the sections were incubated at 25° C. for 60 min with the ABC solution from a VECTASTAIN® ABC kit (Vector Labs PK-4000). After sufficient washing with PBS, all the sections were placed into the same glass exposure chamber and exposed to a 3,3′-diaminobenzidine (DAB, Vector SK-4100) reaction. The reaction was stopped after significant color was produced (10 min) and the sections were sufficiently washed with PBS. The sections were then dried thoroughly and dehydrated in a graded ethanol wash (70% for 5 min, 90% for 5 min, 100% for 5 min) and finally washed with 100% xylene for 10 min. The slides were then mounted and cover-slipped using Cytoseal XYL (Stephens Scientific) and visualized with a Nikon Eclipse E600 microscope with identical light exposure conditions. Images were processed with IPLab software (BD Biosciences) and Photoshop 6.0 (Adobe).

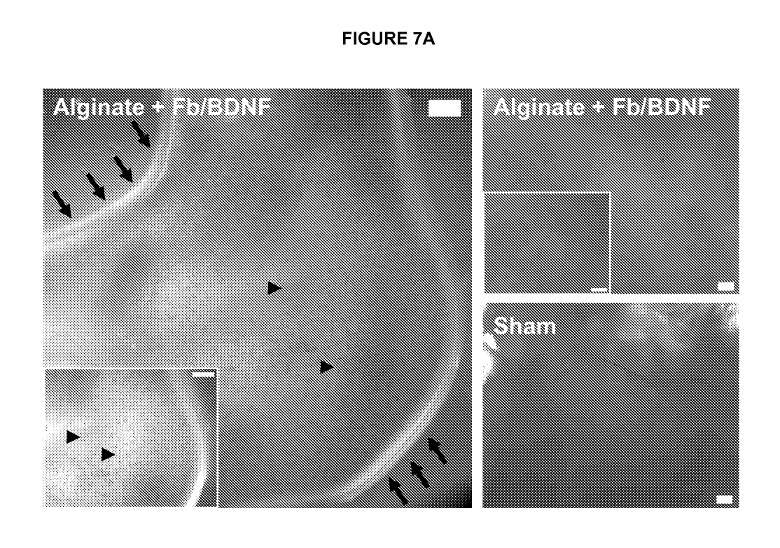

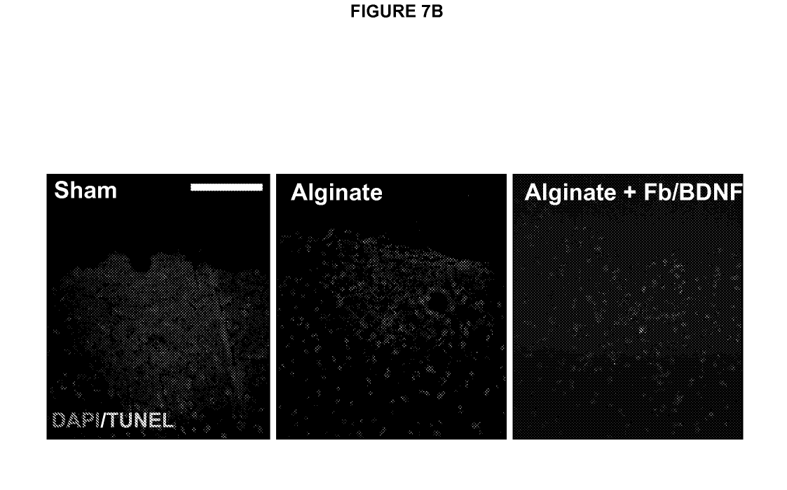

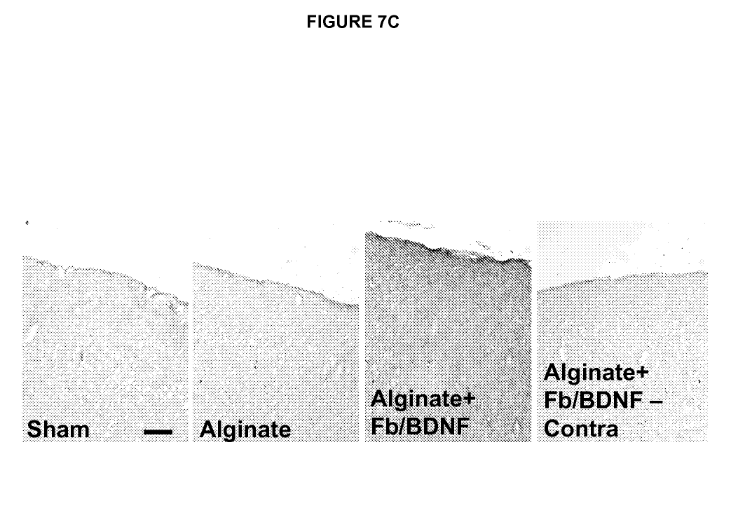

After 7 days, the intact constructs were retrieved and fibroblasts were visualized; the fibroblasts were still viable and producing BDNF, as assessed by β-galactosidase expression, using an X-Gal Staining Kit purchased from Genlantis (San Diego, Calif.) (FIG. 7A). After alginate discs containing Fb/BDNF or Fb/NT3 had been implanted in mice for 7 days, they were recovered and washed with PBS (pH 7.4). Fixing Buffer was then added to each disc and incubated at room temperature for 15 minutes. The Fixing Buffer was then removed and the disc was then washed sufficiently with PBS. X-Gal solution was then added to each disc and incubated at 37° C. for 18 hours to ensure proper staining. The X-Gal solution was then removed and the discs were washed with PBS. The discs were observed and photographs of the discs were taken using an Olympus IX71 fluorescence microscope. Images were processed using SPOT image software to adjust intensity levels.

At the site of implantation, we did not detect any evidence of cell death, suggesting that the substrate was safe for implantation (FIG. 7B). Moreover, elevated levels of BDNF immunoreactivity were detected in the cortical tissue adjacent to the alginate substrates containing encapsulated Fb/BDNF, but not on the contralateral side of the same mouse, or in mice with a sham operation or a transplanted fibroblast-free alginate substrates (FIG. 7C).

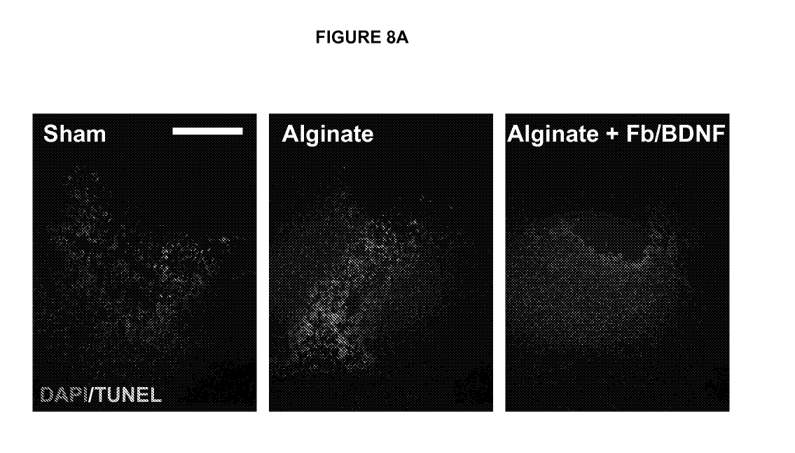

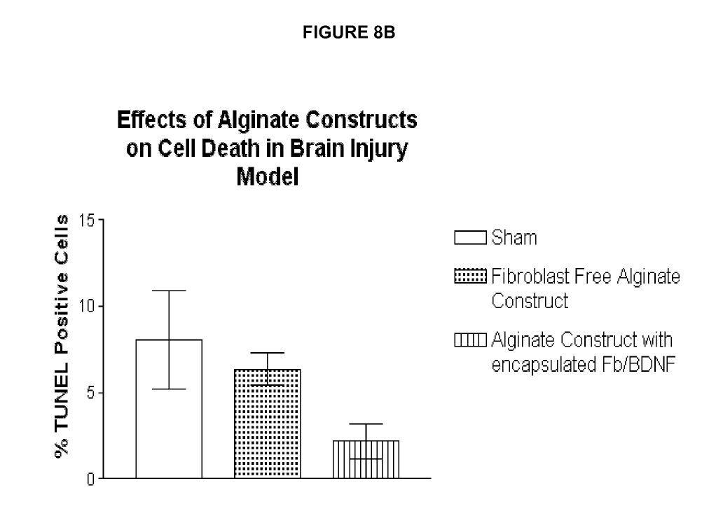

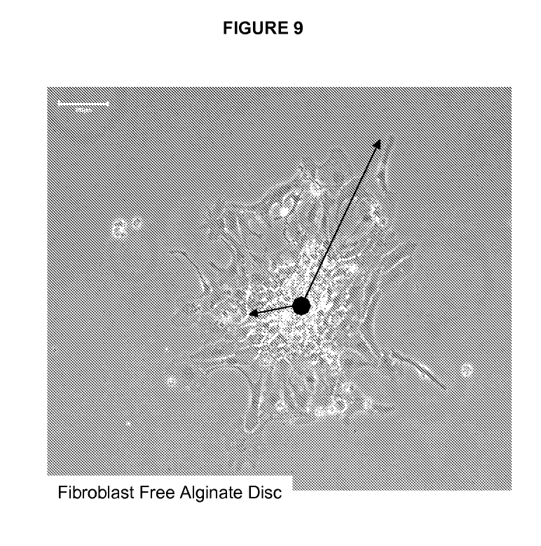

When alginate constructs containing encapsulated Fb/BDNF were transplanted into the cranial cavity of mice with a simulated brain injury, a reduced number of damaged cells was observed. (FIGS. 8A & 8B). The brain injury is a stab wound induced by a Hamilton syringe place into the cortex after the skull is exposed. It is an efficient way of inducing both cell damage and inflammation, cannonical hallmarks of any CNS (brain) injury. We measure damaged cells by the terminal DNA end-nick reaction (TUNEL). This does not measure necrosis but it would be possible to do so by DNA morphology analysis and a series of other reactions. We've previously used this technique before to assess glial differentiation after injury in the cortex. Coksaygan, et al., J Neurosci Res. 2007 Aug. 1; 85(10):2126-37. The percentage of damaged cells for brain injury model mice with a transplanted alginate disc with encapsulated Fb/BDNF was significantly lower than those with a brain injury and a sham implant (damaged cells after implantation of Fb/BDNF construct: 2.2±1.0%, Sham: 8.1±2.8%; p<0.05). Fibroblast free alginate constructs significantly reduced the number damaged cells as compared to a sham (damaged cells after implantation of fibroblast free alginate construct: 6.4±0.9%; Sham: 8.1±2.8%; p<0.05). These results suggest that these alginate constructs can be successfully transplanted in vivo, where they deliver BDNF to the adjacent brain tissue and help to reduce the severity of the sustained injury.

### Example 13

### Formation of Chondroitinase ABC Aggregates

Stable cross-linked aggregates of the enzyme chondroitinase ABC are prepared using the following method. Chondroitinase ABC (0.5 ml enzyme stock solution) is dissolved in 1 ml KH₂PO₄/NaOH buffer (100 mM, pH 7). To this solution was added 1 ml of a 55% (w/v) (NH₄)₂SO₄ solution in the KH₂PO₄/NaOH buffer and 80 μl glutaraldehyde (25% w/v in water. The mixture was stirred at 4° C. for 17 h. A 3 ml aliquot water is added and the mixture is centrifuged to collect the precipitate that forms. The supernatant was decanted and the residue washed 3 more times with water, centrifuged, and decanted; and the residue may be stored at −20° C. Cao et al., Organic Letters, 2000 (2), p. 1361-1364; Lopez-Serrano et al., Biotechnol Letters 2002, (24) p. 1379-1384. For use, the enzyme preparation was dispersed using a magnetic stirrer and stored in 5 ml water at 4° C.

### Example 14

### Alternative Method for Producing Chondroitinase-Modified Alginate

Chondroitinase ABC enzyme was covalently attached to the alginate using an adaptation of aqueous carbodiimide chemistry,(Hermanson G T. Bioconjugate techniques. San Diego, Calif.: Academic Press; 1996. p 169-176) resulting in the formation of an amide bond between the carboxylic acid groups of the alginate and the amine groups on lysines on the Chondroitinase ABC. Alginate was dissolved in MES buffer (0.1 M MES, 0.3 M NaCl, pH 6.5) to obtain a 1% (w/v) solution. 1-ethyl-3-(3-dimethylaminopropyl) carbodiimide (EDC) was added to activate the carboxylic acid groups of the alginate. The amount of EDC added was such that 5% of the carboxylic acid groups of the alginate are molar ratio 1:2 to EDC (28 mg sulfo-NHS/g alginate). The solution was stirred for 15 min to allow the activation of the carboxylic acid groups, following which the appropriate amount of chondroitinase ABC was added. The conjugation reaction proceeded for 24 h at room temperature under gentle stirring. The reaction mixture was then dialyzed for 4 days against about 20 liters of deionized water to remove buffer salts, reaction byproducts, and unreacted enzyme using Spectra/Por dialysis tubing (MWCO 3500). The purified enzyme-alginate conjugate solution was transferred to 50 mL polypropylene tubes, and lyophilized. The final fibrous product was then stored in airtight tubes at 20° C. for use either as substrates for construct fabrication, or as a final coating over a PLO or PLL coated construct.

### Example 15

### Dextran-Modified Chondroitinase

Chondroitinase was chemically stabilized using a dextran spacer arm by a modification of a method described by Maksimenko et al., European Journal of Pharmaceutics and Biopharmaceutics 51:33-38 (2001). Aldehyde dextran was obtained by partial oxidation (50%) of dextrans with periodate. About 2.18 g of sodium periodate were added to a 50 mL solution of dextran (33.33 mg/mL) in distilled water. After 2 hours the oxidized dextrans were dialyzed extensively against distilled water at 4° C. Covalent attachment of chondroitinase ABC (10 μM) to this aldehyde dextran (20-200 μM) was carried out at 4° C. for 30 minutes-18 h using 0.15 M NaCl, 0.1 M phosphate buffer pH 8.3. The resulting Shiff base adduct was reduced by sodium borohydride (10 mg/l) at 4° C. for 30-40 minutes, and isolated by gel filtration on Sephadex G100, or ultrafiltration (Amicon XM-100 membrane), and lyophilized.

### Example 16

### Alternative Method of Producing Dextran-Modified Chondroitinase

Chondroitinase ABC was dissolved in 1 ml of ice cold PBS or NaCl-free HEPES buffer, pH 7.2. Varying amounts of dextran aldehyde and sodium cyanoborohydride were added to the solution, which was then mixed and incubated at 4° C. overnight. Tris•HCl (0.5 ml, 1.0 M, pH 7.2) was added to the mixture and left to incubate at 4° C. for 1-2 hours. The entire mixture was dialyzed against water (12-14 kDa MWCO) at 4° C. overnight, and then lyophilized and stored at −20° C. Mateo et al., Biotechnol Bioeng. 2004 May 5; 86(3):273-6.

### Example 17

### Chondroitinase ABC Stabilized with Bifunctional Gluteraldehyde

Chondroitinase ABC was dissolved in 1 ml acetone 100 mM phosphate buffer (pH 7.0). Ice cold acetone (3 ml) and 80 μl glutaraldehyde were added, and the mixture was stirred at 4° C. for 17 h. Ice cold acetone (1 ml) was added and the mixture was then centrifuged at 8000×g for 10 minutes. The pellet was washed 3 more times with acetone, left to air-dry, and stored at −20° C. Cao et al., supra.; Lopez-Serrano et al., supra.

### Example 18

### Addition of Chondroitinase ABC to Alginate Substrates

Chondroitinase ABC and/or stabilized chondroitinase ABC aggregates encapsulated within the substrate was tested for stability and the ability to diffuse from the substrate into HEPES (4-(2-hydroxyethyl)-1-piperazine ethane sulfonic acid) buffer by measuring enzyme activity over time. Aliquots of release buffer were incubated with chondroitin-6-sulfate, sodium acetate and Tris-HCl, pH 8.0 at 37° C. for 20 min, at which time the reaction was stopped by heating for 1 min at 100° C. After dilution with 50 mM HCl, the absorption at 232 nm was read against a blank.

The pre-formed alginate fibers (above) were coated with PLO at a concentration of 0.5 mg/ml of alginate for 6 minutes. The PLO solution used was 6 times the volume of alginate. The discs and fibers were washed with HEPES buffer to remove any unreacted PLO. The coating time and PLO molecular weight was adjusted to achieve optimal diffusion rates when chondroitinase ABC is utilized. Aliquots of laminin were made by diluting 25 μl laminin in 975 μl culture medium (DMEM+serum replacement+antibiotic) if the alginate contained fibroblasts, or in HEPES or PBS if they do not. Alginate discs and fibers are coated in 1 ml of this laminin mixture/ml of alginate overnight, and washed in HEPES immediately before use.

### Example 19

### Use of Alginate Substrates in Spinal Cord Injury

SCI surgery is performed according to the procedures outlined by Tobias, et. al., J Neurotrauma. 2005 January; 22(1):138-56 and Liu, et al., J Neurosci. 1999; 19(11):4370-87. All procedures follow the NIH guidelines for the care and use of laboratory animals and are approved by the university's Institutional Animal Care and Utilization Committee.

Experimental groups of optimized neurotrophic factor containing substrate, FB/NF (NF may be FB/BDNF, FB/NT-3 or FB/BDNF plus FB/NT-3) and Neural Progenitor (NPCs) are:
1) alginate substrate containing encapsulated FB/NF (n=12)
2) alginate substrate seeded with NPCs (n=12)
3) alginate substrate seeded with NPCs containing encapsulated FB/NF (n=12)
4) alginate substrate containing encapsulated FB/NF and chondroitinase ABC (n=12)
5) alginate substrate seeded with NPCs containing encapsulated FB/NF and chondroitinase ABC (n=12)
6) alginate substrate containing chondroitinase ABC (n=12)
7) alginate substrate seeded with NPCs containing chondroitinase ABC (n=12)
8) cell-free and chondroitinase-free alginate substrate (n=12).

Briefly, female Sprague-Dawley rats are anesthetized and receive a partial hemi-section at the C3/C4 spinal cord segment. The alginate substrate is placed into the lesion cavity, and the dura, muscle, and skin is sutured closed. After surgery, rats are observed until fully awake, being kept on heating pads, before return to the home cages.

To study the effect of the alginate substrates on SCI, rats are deeply anesthetized at 8 weeks post-operation (5 days for chondroitinase ABC), and then transcardially perfused with physiological saline and then 4% paraformaldehyde in phosphate buffered saline (PBS). The spinal cords are removed, washed in PBS and cryoprotected in 30% sucrose for 48-72 hrs at 4° C. Spinal cord tissue is frozen with O.C.T. compound, serially cut into 20 μm sections on a freezing microtome, and processed for Nissl staining and fluorescence immunocytochemistry. See, Tobias , et al., J Neurotrauma. 2005 January; 22(1):138-56 and Liu, et al., J Neurosci. 1999; 19(11):4370-87. Scarring at graft-host interface is monitored using antibodies to GFAP (Glial fibrillary acidic protein). Chondroitin sulfate proteoglycan (CSPG) near the injury is observed with and without chondroitinase releasing alginate using the Chondroitin Sulfate antibody CS-56 (Sigma). Core proteins were measured after removing the GAG side chain use the 2-B-6 antibody. This informs us about the successful breakdown of CSPG as a barrier to axonal growth. The results of chondroitinase treatment, neurotrophic factor release, and NPC growth at 3-5 days post implantation of the alginate, are studied.

Axonal regeneration is studied using anatomical tract tracing procedures (using biotinylated dextran amine, BDA). See, Dolbeare, et al., J. Neurotrauma 2003; 20(11):1251-61.

In addition to morphological and immunohistochemical methods, behavioral methods are used to assess the effect of alginate substrates. Three behavioral tests are performed according to procedures outlined by Tobias, et al., J Neurotrauma. 2005 January; 22(1):138-56 and Liu, et al., J Neurosci. 1999; 19(11):4370-87. Open-field locomotion: Post-operation hindlimb function is evaluated during open field locomotion using the Basso, Beattie and Bresnahan (BBB) locomotor rating scale. See, Dolbeare, et al., J. Neurotrauma 2003; 20(11):1251-61. Rats are trained preoperatively to locomote in a plastic enclosure while two independent observers rate the subject's locomotor ability using the 21-point BBB scale. This test is performed weekly post-operation.

The BBB scale ranges from 0 (no observable hindlimb movement) to 21 (consistent plantar stepping and coordinated gait, consistent toe clearance, predominant paw position is parallel throughout stance, and consistent trunk stability; tail consistently up). See, Basso, et al., Exp Neurol. 1996 June; 139(2):244-56.

Post-operation forelimb function is evaluated while a rat spontaneously explores a vertical clear Plexiglas cylinder. The testing session is videotaped and scored by an independent observer at a later date. The following behaviors is scored: a) independent use of the right or left forelimb to initiate a weight-shifting movement, to contact the cylinder wall during a full rear, or to regain center of gravity while moving laterally while in a vertical position along the wall; b) simultaneous use of both forearms to contact the cylinder wall during a full rear and for lateral movements in a vertical position along the wall 35. Further analysis includes forelimb locomotor rating with a scale devised at our collaborators institution (Drexel College of Medicine). See, Himes, et al., Neurorehabilitation and Neural Repair 2006; 20(2):278-96.

### Example 20

### Dorsal Root Ganglia Collection in SCI

In all cases without the use of NPCs, optimization of the substrates in vitro was performed using dorsal root ganglia to determine neurite growth.

Fertilized white Leghorn eggs (Charles River Laboratories, Wilmington, Mass.) were incubated in a humidified incubator at 37° C. for 9-10 days before use. Eggs containing E9 or E10 embryos were opened and the embryos placed in a sterile Petri dish for dissection. The thoracic cavity of the embryo was opened and the organs removed to expose the spine and dorsal root ganglia (DRGs). The DRGs were removed with dissecting forceps and placed in a 12-well culture dish with prewarmed (37° C.) DMEM.

For bioassay of the BDNF being released from FB/BDNF, DRGs were grown on culture plates in the presence of alginate discs or fibers containing encapsulated FB/BDNF with culture medium (DMEM+serum replacement+antibiotic/antimycotic). DRG neurite outgrowth was observed after 24-48 hours and neurites were measured using Scion Image 4.02 (Scion Corporation, Frederick, Md.) or SPOT (Diagnostic Instruments Inc.).

### Example 21

### Regular Growth and Differentiation in Mouse Neuroblastoma (NB2a) Cells Using Alginate Substrates

Mouse neuroblastoma (NB2a) cells, were cultured in 100 mm culture plates with DMEM (without L-Glutamine), 10% FBS, 2 mM L-Glutamine, and 2 mM Antibiotic/antimycotic at 37° C. and 5% CO₂. The cells were passaged at approximately 70-80% confluency. NB2a cells were harvested from culture using 0.25% trypsin, centrifuged, counted and resuspended in serum free medium made up of DMEM, 10% serum replacement, and 2 mM L-Glutamine. The cells were then seeded on plain alginate substrate coated with laminin as previously described but with the omission of fibroblasts. The final concentration of NB2a cells is 500,000 cells/ml of alginate. After 24 hours the growth medium was replaced with differentiation medium that consists of serum free medium plus 0.1% FBS plus lOμM Dibutryl cyclic adenosine monophosphate (DbcAMP). The differentiation medium was replaced everyday to replenish the DbcAMP.

Mouse neuroblastoma (NB2a) cells, were cultured in 100 mm culture plates with DMEM (without L-Glutamine), 10% FBS, 2 mM L-Glutamine, and 2 mM Antibiotic/antimycotic at 37° C. and 5% CO₂. The cells were passaged at approximately 70-80% confluency. NB2a cells were harvested from culture using 0.25% trypsin, centrifuged, counted and resuspended in serum free medium made up of DMEM, 10% serum replacement, and 2 mM L-Glutamine. The cells were then seeded on the fibroblast-containing alginate substrate coated with laminin as previously described. The final concentration of NB2a cells was 500,000 cells/ml of alginate. Medium was then replaced everyday in order to observe cell proliferation and differentiation.

Alginate discs with encapsulated Neurotrophic factor (BDNF or NT3) producing fibroblasts were prepared using the previously described technique. The substrates were then seeded with undifferentiated mouse neuroblastoma (NB2a) cells. Normally, NB2a cells require the specific differentiation factor, Dibutryl cyclic adenosine monophosphate, in order to extend neurites. As an experimental test of the substrate, this differentiation factor was not added to the media and the cells were observed for 7 days. Unexpectedly, extensive neurite extension from NB2a cells attached to fibroblast encapsulated alginate substrates without the presence of differentiation medium is observed. However, when neurotrophic factor producing fibroblasts were not encapsulated in the alginate substrate, no neurite extension was observed. Neurite extension from NB2a cells on an alginate substrate with encapsulated neurotrophic factor producing fibroblasts was comparable to NB2a cells grown on plain laminin coated alginate discs with supplemented differentiating factor. This leads to the discovery that alginate encapsulated neurotrophic factor producing fibroblasts promote the differentiation of NB2a neuroblastoma cells and can cause neurite extension without the incorporation of differentiation factors. Control studies were done where NB2a cells supplemented with exogenous BDNF or NT3. The aforementioned study did not promote any neurite extension, leading to the conclusion that the neurotrophic factor producing fibroblasts are essential, and when encapsulated in alginate, enhance and aid in cell differentiation and neurite extension.

What is claimed:

1. A substrate comprising biocompatible gel and at least one mesenchymal stem cell, neural progenitor cell, or genetically modified fibroblast.

2. The substrate of claim 1, wherein the biocompatible gel is alginate.

3. The substrate of claim 1, further comprising a non-cellular therapeutic agent.

4. The substrate of claim 1, wherein the genetically modified fibroblast produces at least one therapeutic agent.

5. The substrate of claim 1, in the configuration of a fiber having an average diameter of about 50-1000 μm.

6. The substrate of claim 1, further comprising a coating.

7. The substrate of claim 1, wherein a sharp instrument is partly embedded in one of the ends of the substrate.

8. A method of molding a solution comprising filling a mold with a solution of biocompatible gel and at least one mesenchymal stem cell, neural progenitor cell, or genetically modified fibroblast; and a cross-linking agent to produce a shaped body.

9. The method of claim 8, wherein the shaped body has circular or linear indentations.

10. The method of claim 8, wherein the biocompatible gel is alginate.

11. The method of claim 8, wherein the cells are present in a gradient across the substrate.

12. The method of claim 8 further comprising a therapeutic agent.

13. The method of claim 8, wherein the genetically modified fibroblast produces at least one therapeutic agent.

14. The method of claim 8, further comprising seeding the solution with NPC.

15. The use of a substrate comprising biocompatible gel and at least one mesenchymal stem cell, neural progenitor cell, or genetically modified fibroblast for the preparation of a medicament for the treatment of repairing damaged nervous tissue in an animal.

16. The use of claim 15, wherein the biocompatible gel is alginate.

17. The use of claim 15, wherein the genetically modified fibroblast produces at least one therapeutic agent.

18. The use of claim 15, wherein the substrate is in the configuration of a fiber having an average diameter of about 50-1000 μm.

19. The use of claim 15, wherein the substrate further comprises a coating.

20. The use of claim 15, wherein the substrate is placed in proximity to the damaged nervous tissue.
